(19) United States
(12) Patent Application Publication
(10) Pub. No.: US 2020/0049288 A1
(43) Pub. Date: Feb. 13, 2020

(54) Functionalized Unitary Molded Manifolds For Closed Fluid Handling Systems

(71) Applicant: Invetech, Inc., San Diego, CA (US)

(72) Inventors: David Thomas Kneen, Northcote (AU); Robert Alister Neil, Wantirna South (AU)

(21) Appl. No.: 16/539,355

(22) Filed: Aug. 13, 2019

Related U.S. Application Data
(60) Provisional application No. 62/718,155, filed on Aug. 13, 2018.

(51) Int. Cl. F16L 29/00; F16K 17/16; F16K 7/04; A61M 39/18; A61M 39/22

(52) U.S. Cl. CPC F16L 29/00; F16K 17/16; A61M 39/221; A61M 39/18; F16K 7/04

(57) ABSTRACT

A connector system having for joining tubes having an overmolded or bonded connector or manifold incorporating specialized features. The overmolded or bonded connector includes a tubular body, an input portion, at least one output portion, an internal fluid passageway, and at least one specialized feature such as a valve or sensor integrally molded or embedded therein. The connection system may be pre-sterilized, disposable and made for single-time usage in a closed fluid handling system.

### CROSS-REFERENCE TO RELATED APPLICATION

This application claims the benefit under 35 U.S.C. § 119(e) of the earlier filing date of U.S. Provisional Patent Application No. 62/718,155 filed on Aug. 13, 2018 the disclosure of which is incorporated by reference herein.

### BACKGROUND

Processing of fluids including liquids, emulsions, slurries, gases and mixtures of these that must be isolated from the outside world often takes place within closed disposables. The closed disposable serves to protect the material from contamination by the outside world, protect the outside world from contamination by the material, and provide a single-use environment for processing of that material that avoids the need for cleaning between process runs. Typical applications include the preparation of pharmaceutical, biological and/or hazardous materials such as cell cultures for cell therapy, gene therapy and regenerative medicine, virus materials including viral vectors, bacterial cultures and their extracts, media and reagent handling, active pharmaceutical ingredients such as protein preparations, and hazardous or poisonous materials such as radioactive dyes.

Closed disposables are widely used in the industry, and typically comprise a series of bags, tubing and other components, coupled together into a single closed unit, and often validated as sterile and free from leaks. Manipulation of the materials inside the closed disposable happens without breaching the disposable barrier—for example heating/cooling, pumping, mixing/separating, connecting/disconnecting, pressurization/vacuum and many other physical manipulations.

A fundamental challenge in the manufacture of closed disposables is the joining of multiple tubes to form a passage, junction or manifold. Typically this is done through barbed connections where tubes are pressed over a molded plastic barb and often retained with an external fitting, bonded connections where tubes are pressed either inside or outside of a fitting and affixed using either a glue or solvent, overmolded connections where tubes are arranged around a removable plug and a material is overmolded around those to form a bonded connection, welded connections where tubes are locally melted and pressed together to form a weld, and other approaches.

Where multiple tubes are joined at a junction, they form a node in the fluidic circuit created. In an ideal fluidic circuit, control operations happen at the nodes—for example valve opening/closing. In each of the methods described for joining tubes above, the node is inaccessible due to the physical structure of junction. Barbed junctions may leak if the nearby tubing is pinched, which could contaminate the product or cause valuable product to be wasted. Overmolded junctions are thicker than the rest of the tubing, due to the additional layer of material that is molded over the tubing. As a result, ovemolded connections require more force to pinch closed, which requires more expensive valves and may cause the tubing to become stuck closed when the valve is released. Hence control operations must be translated away from the junction, typically to an unimpeded section of tube at some distance from the junction. This separation hinders the performance of the fluidic circuit in many ways, including dispense precision, switching capability, carryover and cross-contamination, wastage and recovery, response time and so on. In addition, the physical structure of the junction limits the ability to minimize the size of the disposable, imposes handling challenges such as tangling for complex disposables, and leads to a complex interface between the disposable and any machine that the disposable must interface with.

It is, therefore, an object of the present disclosure to overcome the above problems and others by functionalizing the tubing junction connectors by adding features within the overmolded or bonded body of a fluidic connector. In existing overmolded or bonded connectors, the overmolded or bonded body serves simply to connect the tubes, with the inherent issues described above.

### BRIEF DESCRIPTION OF THE DRAWINGS

Various embodiments of the invention are described herein in by way of example in conjunction with the following figures, wherein like reference characters designate the same or similar elements.

FIG. 1A shows a schematic cross-sectional side view representing an overmolded or bonded connector incorporating a normally open valve feature in an open position:

FIG. 1B shows a schematic cross-sectional side view representing an overmolded or bonded connector incorporating a normally open valve feature in a closed position;

FIG. 2 shows a schematic cross-sectional side view representing an overmolded or bonded connector incorporating a normally unidirectional valve feature;

FIG. 3 shows a schematic cross-sectional side view representing an overmolded or bonded connector incorporating a step-up/down feature;

FIG. 4 shows a schematic cross-sectional side view representing an overmolded or bonded connector incorporating a burstable membrane feature;

FIGS. 5A-5C show schematic cross-sectional side views representing an overmolded or bonded connector incorporating a sealable valve in open (FIG. 5A), scaled (FIG. 5B) and separated (FIG. 5C) positions;

FIG. 6A shows a schematic cross-sectional side view representing an overmolded or bonded connector incorporating a tortuous path mixing feature;

FIG. 6B shows a schematic cross-sectional side view representing an overmolded or bonded connector incorporating an external mixing feature;

FIG. 7 shows a schematic cross-sectional side view representing an overmolded or bonded connector incorporating a gas permeable feature;

FIGS. 8A-8C show schematic cross-sectional side views representing an overmolded or bonded connector incorporating alignment features; square (FIG. 8A), triangular (FIG. 8B) and ridged (FIG. 8C);

FIG. 9 shows a schematic cross-sectional side view representing an overmolded or bonded connector incorporating a window feature:

FIGS. 10A-10B show schematic cross-sectional side views representing an overmolded or bonded connector incorporating heat transfer features: heating (FIG. 10A), and cooling (FIG. 10B);

FIG. 11 shows a schematic cross-sectional side view representing an overmolded or bonded connector incorporating a sensor feature;

FIG. 12 shows a schematic cross-sectional side view representing an overmolded or bonded connector incorporating an identifier feature;

FIGS. 13A-13C show schematic cross-sectional side views representing an overmolded or bonded connector incorporating a bellows feature in first open (FIG. 13A), closed (FIG. 13B) and second open (FIG. 13C) positions;

FIG. 14 shows a schematic cross-sectional side view representing an overmolded or bonded manifold incorporating a stiffener feature;

FIG. 15 shows a schematic cross-sectional side view representing an overmolded or bonded connector incorporating multiple features;

FIG. 16 shows a schematic cross-sectional side view representing an overmolded or bonded manifold incorporating multiple features; and

FIG. 17 shows a schematic cross-sectional side view representing an overmolded or bonded manifold incorporating multiple features including elongated branches.

### DETAILED DESCRIPTION

Embodiments of this disclosure functionalize the tubing junction by adding designed features within the overmolded or bonded body of a fluidic connector. Embodiments of this disclosure provide reduced assembly cost, part count, more compact assemblies, more robust assemblies, greater repeatability of processing through more consistent geometry and lower part-to-part variance, the ability to produce equivalent connectors in multiple materials as suited to the fluid handling operation, reduction of settling and/or dead-spot loss at junctions, and reduced opportunity for leachables/extractables contamination of the fluid. For example, overmolded manifolds according to embodiments of this disclosure can be created in a single molding stem at a lower assembly cost than traditional manifolds which must be assembled manually. Likewise, bonded embodiments of the invention only require minimal assembly after the molding step, to bond the tubes. Embodiments in which one or more functions (e.g., valving, sealing, mixing, etc.) are embedded in the manifold have a lower part count and cost than traditional manifolds that require additional parts to implement the functions. Embodiments of this disclosure provide more robustness, more consistent geometry, and lower part-to-part variance because the functional components are integrated or embedded directly into the manifold as part of a single well-controlled manufacturing step, as opposed to traditional manifolds where multiple joints are created in a largely uncontrolled manual assembly process. As a result, manifolds manufactured according to embodiments of this disclosure are more consistent, stronger, less prone to tangling or handling issues, and have fewer opportunities for failures or leaks.

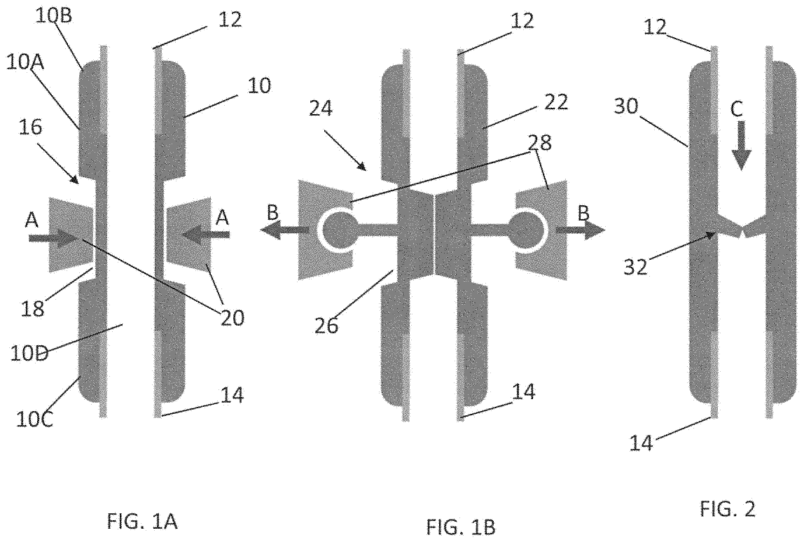

The principles of the illustrated embodiments apply to both overmolded and bonded connectors. Since the connectors are represented schematically in the drawings, FIGS. 1-17 are considered to represent embodiments of both overmolded and bonded connectors or manifolds. As discussed above, overmolded connectors or manifolds are formed by arranging tubes around a removable plug and overmolding a material around them, and bonded connectors or manifolds are formed by pressing tubes either inside or outside of a fitting and affixing using either a glue or solvent.

The overmolded and bonded connectors and manifolds according to embodiments of this disclosure are intended for biotechnology uses and are intended to be pre-sterilized, disposable and made for single-time usage. The overmolded and bonded connectors, manifolds and tubing according to embodiments of this disclosure are formed from, for example, silicone, thermoplastic elastomers (TPF), polyolefins (POF), polyvinyl chloride (PVC), polyethylene (PE), or any other suitable material. The typical pressure range that these are likely to be operated in are from +4 bar −1 bar (−15 p.s.i.) to (+60 p.s.i.).

The below-described specialized features are molded or embedded into the connectors and manifolds forming a unitary piece.

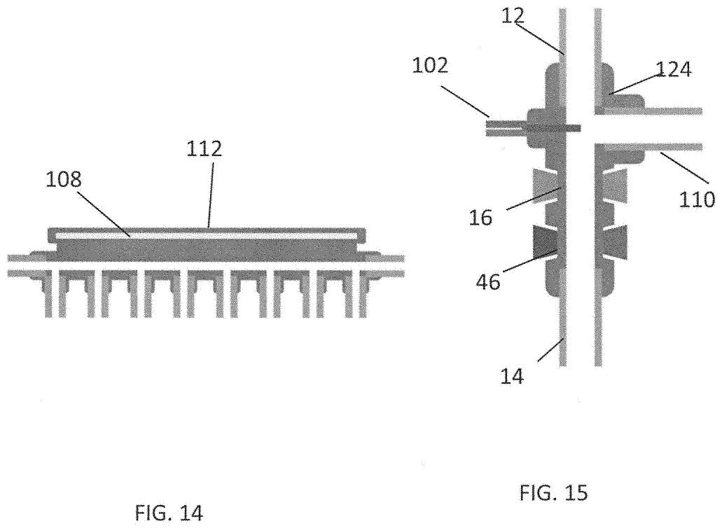

Embodiments are shown in FIGS. 1-13C as overmolded or bonded connectors for two tubes incorporating specialized features. The principles of the embodiments of FIGS. 1-13C also apply to multi-tube connectors and manifolds such as those shown in FIGS. 14-17.

FIGS. 1A-2 show embodiments of overmolded or bonded connectors incorporating valving.

Referring to FIG. 1A, an overmolded or bonded connector 10 joining two tubes 12 and 14 is shown. Overmolded or bonded connector 10 includes a tubular body 10A having a first end 10B overmolded or bonded to tube 12 forming an overmolded or bonded connection, a second end 10C overmolded or bonded to tube 14 forming an overmolded or bonded connection, and a fluid passageway 10D therebetween. Likewise, the overmolded or bonded connectors of the embodiments of 1B-17 have tubular bodies, first and second ends and fluid passageways but are not provided references.

Overmolded or bonded connector 10 incorporates a normally open valve 16. Normally opened valve 16 includes a normally open pinch portion 18 of decreased outer diameter structured to interface with an external pinch valve mechanism 20. To close normally opened valve 16, external pinch valve mechanism 20 pinches pinch portion 18 of overmolded or bonded connector 10 to close normally open valve 16 by moving in directions A.

Referring to FIG. 1B, an overmolded or bonded connector 22 joining two tubes 12 and 14 is shown. Overmolded or bonded connector 22 incorporates a normally closed valve 24. Normally closed valve 24 includes a normally closed pinch portion 26 structured to interface with an external pinch valve mechanism 28. To open normally closed valve 24, external pinch valve mechanism 28 retracts normally closed pinch portion 26 of overmolded or bonded connector 22 to open normally closed valve 24 by moving in directions B.

Referring to FIG. 2, an overmolded or bonded connector 30 joining two tubes 12 and 14 is shown. Overmolded or bonded connector 30 incorporates a unidirectional valve 32. Unidirectional valve 32 includes an internal passive feature driven by flow allowing flow in a single direction C.

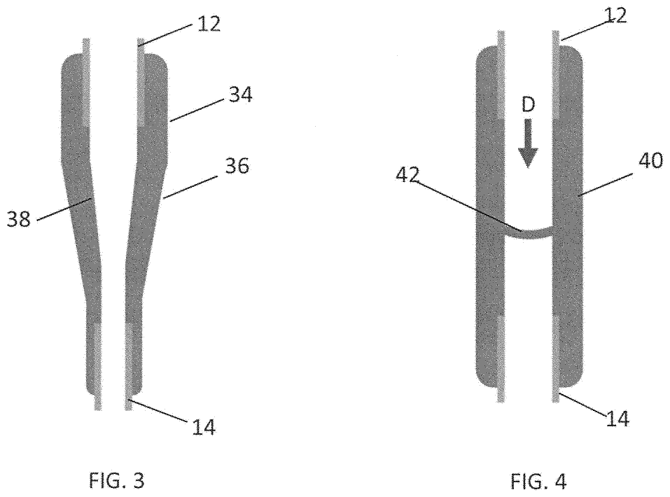

Referring to FIG. 3, an overmolded or bonded connector 34 joining two tubes 12 and 14 is shown wherein tube 12 has a larger bore diameter than tube 14. Overmolded or bonded connector 34 incorporates step-up/down feature 36 including a portion 38 of gradual change of internal bore diameter of step-up/down feature 36 such the diameter of the step-up/down feature 36 decreases between the connection with tube 12 and the connection with tube 14.

Referring to FIG. 4, an overmolded or bonded connector 40 joining two tubes 12 and 14 is shown. Overmolded or bonded connector 40 incorporates a burstable membrane 42 of characterized burst strength acting as a one-time normally closed valve.

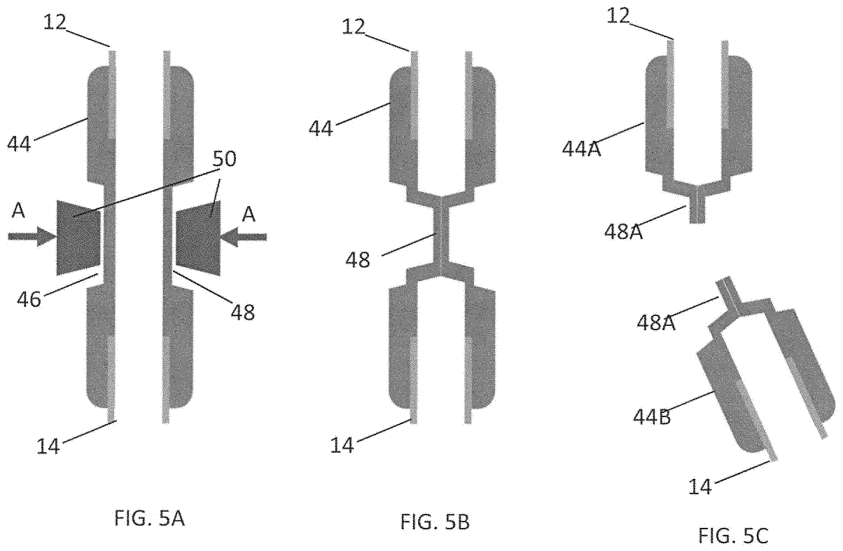

Referring to FIGS. 5A-5C, an overmolded or bonded connector 44 joining two tubes 12 and 14 is shown. Overmolded or bonded connector 44 incorporates a permanently sealable and disconnectable valve 46 which is normally open. Permanently sealable and disconnectable valve 46 includes a sealable pinch portion 48 of decreased outer diameter structured to interface with a sealer 50 such as a heat or radiofrequency sealer. As shown in FIG. 5A, to permanently seal valve 46, sealer pinches sealable pinch portion 48 in direction A to close the normally open valve 46 by moving in directions A to form a sealed valve 46 by sealing pinch portion 48 (FIG. 5B). After sealing, the overmolded or bonded connector 44 may be separated into two pieces 44A, 44B by cutting or tearing mid-sealed pinch portion 48. (FIG. 5C) to form sterile, permanent seals 48A, 48B.

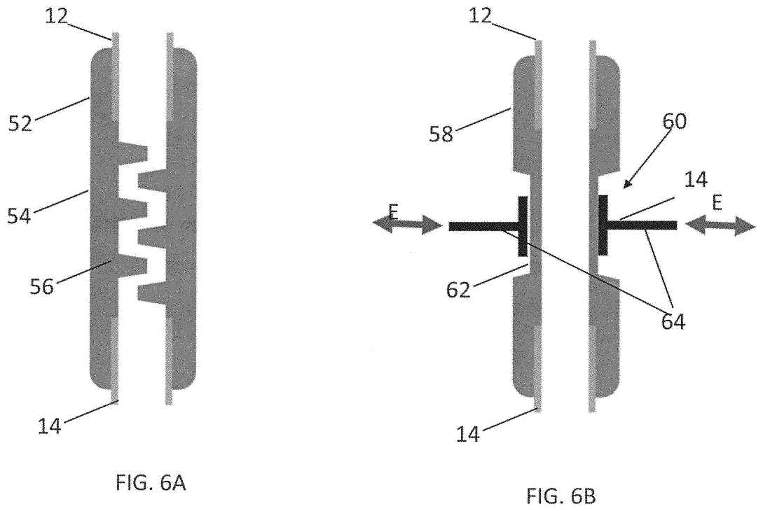

FIGS. 6A-6B show embodiments of overmolded or bonded connectors incorporating mixing or homogenizing features.

Referring to FIG. 6A, an overmolded or bonded connector 52 joining two tubes 12 and 14 is shown. Overmolded or bonded connector 52 incorporates a passive internal mixing feature 54 such as a tortuous path, shear or flow-disrupting mixing features. In this embodiment, passive internal mixing feature 54 includes a series of internal, offset projections 56 forming a tortuous path.

Referring to FIG. 68, an overmolded or bonded connector 58 joining two tubes 12 and 14 is shown. Overmolded or bonded connector 58 incorporates an active mixing feature 60. In this embodiment, active mixing feature 60 includes a mixer interface portion 62 which is structured to interface with an external mixing device 64 such as a vibratory, sonic or ultrasonic mixer which operates on the mixer interface portion 62 in the back and forth directions E, for example.

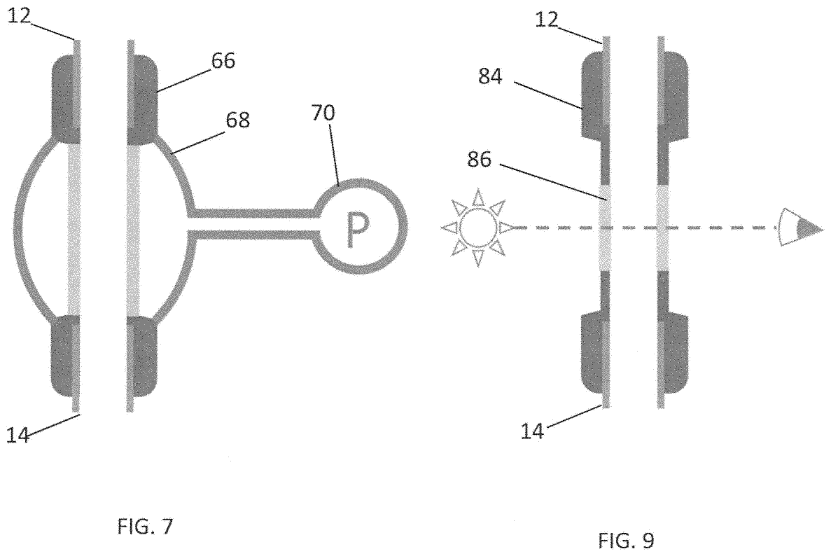

Referring to FIG. 7, an overmolded or bonded connector 66 joining two tubes 12 and 14 is shown. Overmolded or bonded connector 66 incorporates a gas-permeable portion 68 formed of a gas permeable material allowing controlled gas transfer when coupled with a pressure or vacuum source 70.

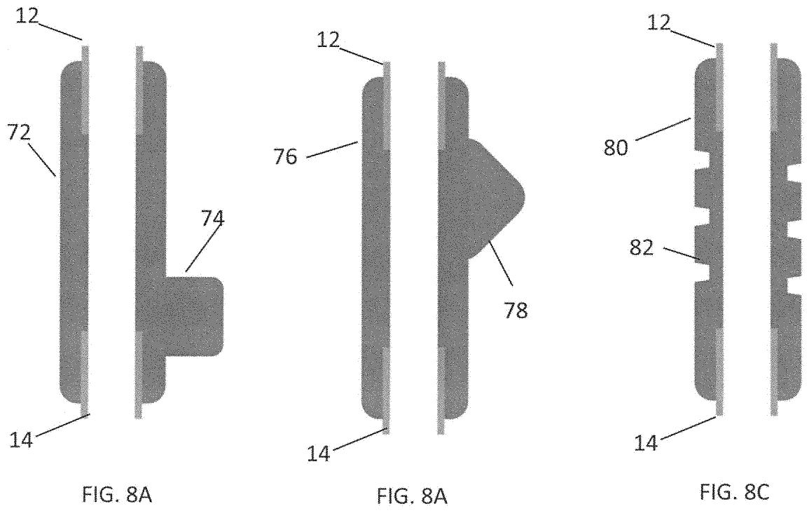

FIGS. 8A-8C show embodiments of overmolded or bonded connectors incorporating external alignment features to ensure poke-yoke loading, enable easy insertion into machines, auto-alignment and to ensure simple handling for operators. Referring to FIG. 8A, an overmolded or bonded connector 72 having a square-shaped external alignment feature 74 is shown.

Referring to FIG. 8B, an overmolded or bonded connector 76 having a triangular-shaped external alignment feature 78 is shown.

Referring to FIG. 8C, an overmolded or bonded connector 80 having an external ridged alignment feature 82 is shown.

Referring to FIG. 9, an overmolded or bonded connector 84 joining two tubes 12 and 14 is shown. Overmolded or bonded connector 84 incorporates a window 86 configured for optical inspection and analysis of, for example, turbidity, refractive index or the presence/absence of fluid. Window 86 may also be configured to allow, for example, laser interrogation, camera visualization.

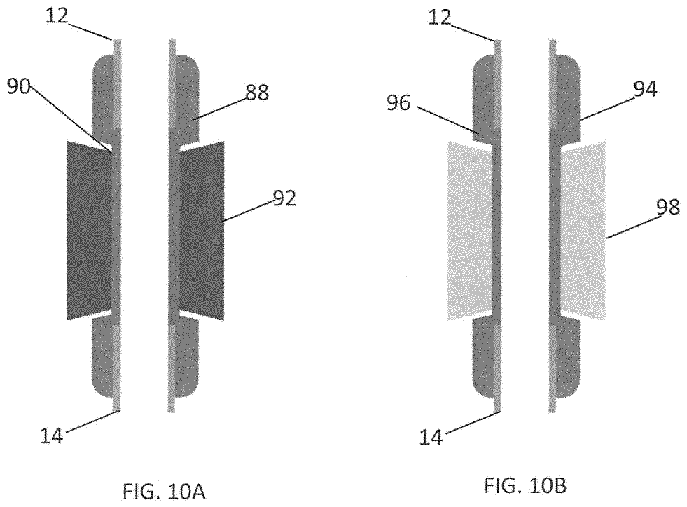

FIGS. 10A-10B show embodiments of overmolded or bonded connectors incorporating heating/cooling features for temperature maintenance and/or change during, for example, endothermic or exothermic mixing.

Referring to FIG. 10A, an overmolded or bonded connector 88 joining two tubes 12 and 14 is shown. Overmolded or bonded connector 88 incorporates a heat transfer portion 90 of decreased outer diameter allowing for the application of an external heat source 92

Referring to FIG. 10A, an overmolded or bonded connector 94 joining two tubes 12 and 14 is shown. Overmolded or bonded connector 94 incorporates a heat transfer portion 96 of decreased outer diameter allowing for the application of an external cooling source 98.

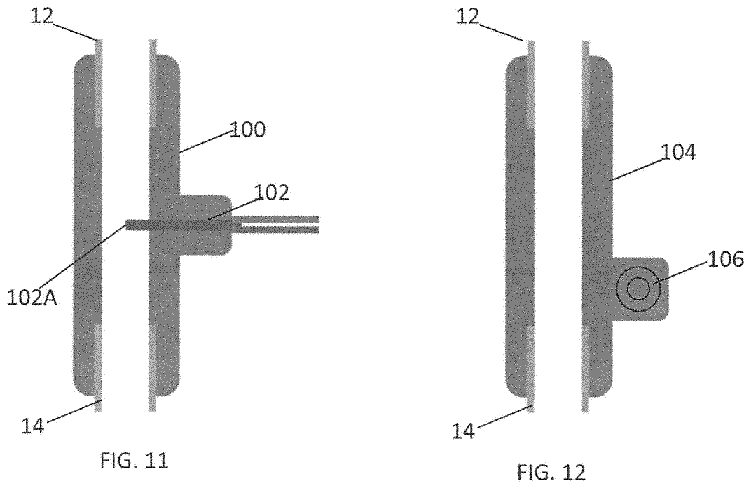

Referring to FIG. 11, an overmolded or bonded connector 100 joining two tubes 12 and 14 is shown. Overmolded or bonded connector 100 incorporates a sensor 102 such as a sensor for measuring flow, pressure, temperature, oxygen, pH and carbon dioxide. As shown in FIG. 10, sensor 102 may be embedded into overmolded or bonded connector 100 such that a portion thereof is exposed to the fluid path within overmolded or bonded connector 100.

Referring to FIG. 12, an overmolded or bonded connector 104 joining two tubes 12 and 14 is shown. Overmolded or bonded connector 104 incorporates embedded identifiers 106 such as color tags, radio-frequency identification (RFID), or other labelling identifying the connector such as Bluetooth low energy (BLE) technology or Quick Response (QR) codes.

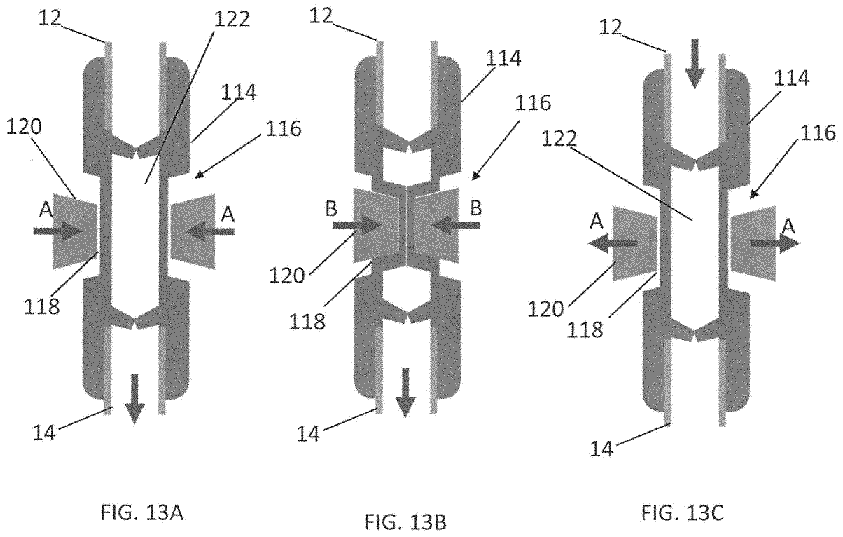

Referring to FIGS. 13A-13C, an overmolded or bonded connector 114 is shown incorporating a bellows feature 116 and two unidirectional valves 32 for metered pumping. As shown in FIG. 13A, overmolded or bonded connector 114 includes a normally open bellows portion 118 of decreased outer diameter structured to interface with an external bellows mechanism 120 which is configured to sequentially compress against bellows portion 118 to expel fluid from a central reservoir 122 formed in between the two unidirectional valves 32 (FIG. 13B) and then retract away from bellows portion 118 to allow central reservoir 122 to be filled (FIG. 13C).

Referring to FIG. 14, an overmolded or bonded manifold 108 is shown having stiffening features 112. Overmolded or bonded connectors are inherently more rigid and less prone to tangling than traditional connectors that do not use overmolding or bonding. The stiffening features 112 may be further added to enhance the inherent rigidity. The incorporation of the other features disclosed herein would also enhance the inherent rigidity.

FIGS. 15-17 show examples of embodiments of overmolded or bonded connectors incorporating more than one feature. The embodiments below are not limited to the illustrated combination of features and one or more of the above-described features may be combined, depending on the desired functionality of the connector. All of the functionality of the different features may be provided within a single compact component.

Referring to FIG. 15, an overmolded or bonded connector 124 joining three tubes 12, 14 and 110 is shown. Overmolded or bonded connector 108 incorporates more than one feature further functionalizing overmolded or bonded connector 108 and allowing the ability to provide a more compact assembly than if the features were positioned on the tubes. In the illustrated embodiment, overmolded or bonded connector 108 includes a sensor 102 such as a pressure sensor, a normally open valve 16 and a heat sealable valve 46, for example.

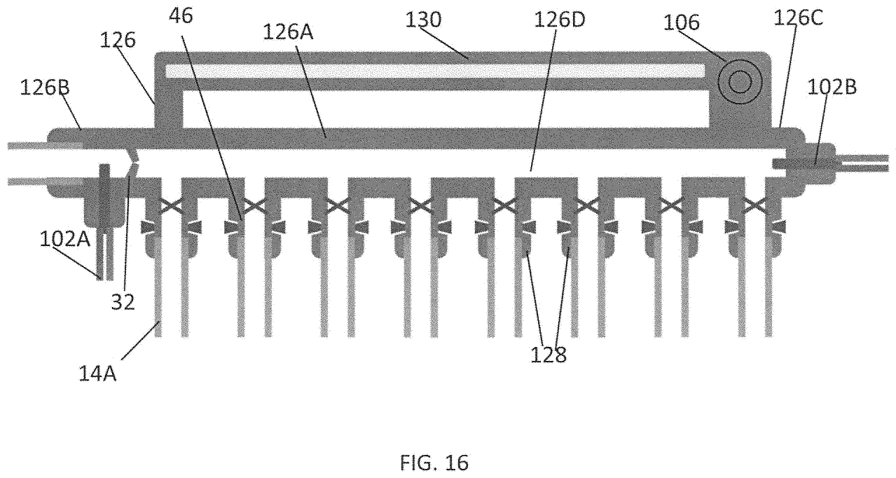

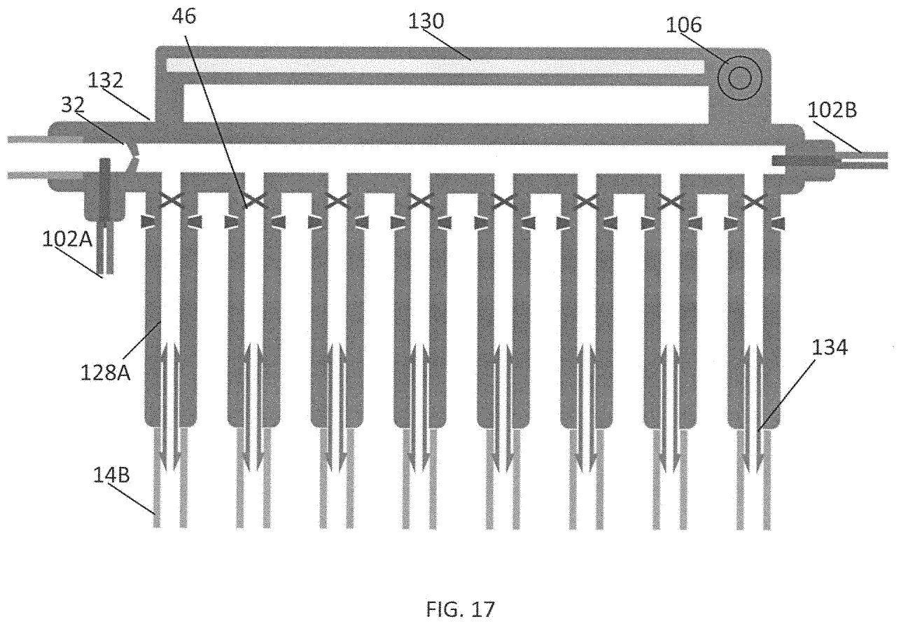

FIGS. 16 and 17 show embodiments of overmolded or bonded connectors in the form of manifolds incorporating more than one integrally molded or embedded feature. The overmolded or bonded manifolds in the illustrated embodiments have at least one input end portion fluidly connected to at least one output portion.

Referring to FIG. 16, an overmolded or bonded manifold 126 is shown having multiple features in a single connector. In this embodiment, overmolded or bonded manifold 126 connects an input bag (not shown) and multiple output bags (not shown) for a filling operation. As shown overmolded or bonded manifold 126 has a tubular body 126A having a first input end 126B overmolded or bonded to a tube 12, a second closed end 126C, and, for example, eight output branches 128 spaced down the length of the tubular body 126A which are each overmolded or bonded to eight respective tubes 14A. A fluid passageway 126D is disposed between first input end 126B and second closed end 126C and is fluidly connected to the output branches 128.

Overmolded or bonded manifold 126 integrally incorporates multiple functions discussed above, for example, a unidirectional valve 32 disposed at first input end 126B, an upstream flowrate sensor 102A disposed at first input end 126B, a downstream pressure sensor 102B disposed at second closed end 126C, a rigid handle 130 with a poke-yoke feature, and an integrated identifier 106 disposed on handle 130. Each branch 128 may further include a permanently sealable and disconnectable pinch valve 46 closing off output to tubes 14A.

In another embodiment, the sections coming oft the overmolded manifold may be longer, and effectively act as short tubes that branch off the manifold. Instead of being directly overmolded into the manifold, the tubes that connect the output bags could be connected to the manifold using barbs or other traditional connectors. This could be useful in scenarios where the materials are incompatible for bonding or overmolding. Like the embodiment of FIG. 16, the embodiment of FIG. 17 shows an overmolded or bonded manifold 132 connecting an input bag (not shown) and multiple output bags (not shown) for a filling operation. Like the embodiment of FIG. 16, overmolded or bonded manifold 132 has, for example, eight branches 128A and integrally incorporates a unidirectional valve 32, an upstream flowrate sensor 102A, an integrated identifier 106, a downstream pressure sensor 102B, and a rigid handle 130 with a poke-yoke feature. Each branch 128A may further include a permanently sealable and disconnectable pinch valve 46. In this embodiment, each branch 128A is elongated so that it can further allow the use of barbed connections 134 for connections with additional tubing 14B where the additional tubing 14B is incompatible with overmolding.

Overmolded or bonded manifold 126, 132 may further include one or more of the features described with reference to from FIGS. 1A-14 in addition to those shown in FIGS. 16 and 17. For example, overmolded or bonded manifold 126, 132 may further include one or more of the following features molded or embedded in, for example, the tubular body, the input portion, and/or the at least one output portion of the overmolded or bonded manifold 126, 132: normally open valve 16, a normally closed valve 24, a step-up/down feature 36, a burstable membrane 42, a mixing feature 54, 60, a gas-permeable portion 68, a window 86, a heat transfer portion 90, 96 for heating or cooling, or a bellows feature 116.

Nothing in the above description is meant to limit the invention to any specific materials, geometry, or orientation of elements. Many part/orientation substitutions are contemplated within the scope of the invention and will be apparent to those skilled in the art. The embodiments described herein were presented by way of example only and should not be used to limit the scope of the invention.

Although the invention has been described in terms of particular embodiments in this application, one of ordinary skill in the art, in light of the teachings herein, can generate additional embodiments and modifications without departing from the spirit of, or exceeding the scope of, the described invention. Accordingly, it is understood that the drawings and the descriptions herein are proffered only to facilitate comprehension of the invention and should not be construed to limit the scope thereof.

What is claimed is:

1. A connector system configured for a closed fluid system comprising:
a connector comprising:
a tubular body having a first end and a second end;
an input portion disposed at the first end;
at least one output portion;
an internal fluid passageway connecting the input portion and the at least one output portion; and
at least one integrated feature integrated into at least one of the tubular body, input portion, or output portion; and
a first tube, wherein the input portion includes a connection selected from an overmolded input connection and a bonded connection connecting the input portion to the first tube;
wherein the at least one output portion includes an output connection configured to connect the at least one output portion to at least one second tube.

2. The connector system of claim 1, wherein the at least one integrated feature is selected from the group consisting of at least one valve, a step-up/down feature, a sensor, an identifier, a burstable membrane, a mixing feature, a poke-yoke feature, a gas-permeable portion, a window, a heat transfer portion for heating or cooling, and a bellows feature.

3. The connector system of claim 1, wherein the at least one output portion comprises a plurality of spaced output branches.

4. The connector system of claim 3, wherein the at least one integrated feature comprises a first valve disposed within the input portion.

5. The connector system of claim 4, wherein the first valve comprises a unidirectional valve.

6. The connector system of claim 4, wherein the at least one integrated feature further comprises at least one second valve disposed in at least one of the output branches.

7. The connector system of claim 6, wherein each second valve comprises a sealable valve.

8. The connector system of claim 1, wherein the integrated feature comprises a first sensor disposed in the input portion.

9. The connector system of claim 8, wherein the first sensor comprises a flowrate sensor.

10. The connector system of claim 8, wherein the at least one integrated feature further comprises a second sensor disposed in the second end of the tubular body.

11. The connector system of claim 10, wherein the second sensor comprises a pressure sensor.

12. The connector system of claim 1, wherein the integrated feature comprises a poke-yoke feature.

13. The connector system of claim 1, wherein the integrated feature comprises an identifier.

14. The connector system of claim 1, wherein the integrated feature comprises at least one step-up down feature disposed in at least one of the input portion and the at least one output portion.

15. The connector system of claim 1, wherein the integrated feature comprises at least one burstable membrane.

16. The connector system of claim 1, wherein the integrated feature comprises a mixing feature disposed in the tubular body.

17. The connector system of claim 1, wherein the integrated feature comprises a gas-permeable portion disposed in the tubular body.

18. The connector system of claim 1, wherein the integrated feature comprises a window disposed in the tubular body.

19. The connector system of claim 1, wherein the integrated feature comprises a heat-transfer portion disposed in the tubular body.

20. The connector system of claim 1, wherein the integrated feature comprises a bellows feature disposed in the tubular body.

21. The connector system of claim 1, wherein each output connection is one of an overmolded and bonded connection to a second tube.

22. The connector system of claim 1, wherein the connector is composed of a material selected from silicone, thermoplastic elastomers (TPE), polyolefins (POF), polyvinyl chloride (PVC), polyethylene (PE), or any other suitable material.
